(19) United States
(12) Patent Application Publication
(10) Pub. No.: US 2020/0297285 A1
(43) Pub. Date: Sep. 24, 2020

(54) Monitoring System and Analysis and Control of Body Activities

(71) Applicant: Shahrokh Ghalavand, San Rafael, CA (US)

(72) Inventor: Shahrokh Ghalavand, San Rafael, CA (US)

(21) Appl. No.: 16/360,813

(22) Filed: Mar. 21, 2019

(51) Int. Cl. A61B 5/00; A61B 5/11

(52) U.S. Cl. CPC A61B 5/7275; A61B 5/1112; A61B 5/1113; A61B 5/1116; A61B 5/445; A61B 5/4094; A61B 2503/12; A61B 5/742; A61B 5/0022; A61B 2562/0219; A61B 2503/08; A61B 2503/06; A61B 5/4088

(57) ABSTRACT

A body monitoring system is designed for patients of varying needs with patients at risk for seizures, epilepsy, stroke, heart attack, bedsores, dementia, mental disorders, Old aged people on elderly, children specifically hyperactive ones. This system provides the user the control by online observation and activities algebra sum table sand database. The system has two warning systems: in APP and cell phone in the emergency. A plurality of sensors measuring angle and movement and acceleration in the XYZ axes in combination with a plurality of GPS sensors and GPS boundary makers and a heart rate sensor is used. Show and control and monitoring positions of the body during resting and the time and place activities and body movement of patients the states and tensions have problems psychological. Online display current status of patient and saving the states and conditions of the patient.

### CROSS-REFERENCE TO RELATED APPLICATIONS

NA

### BACKGROUND OF THE INVENTION

### (1) Field of the Invention

The invention generally relates to body monitoring systems. More particularly, the invention relates to means and methods of using body monitoring components and various systems to diagnose and/or treat illness.

### BRIEF SUMMARY OF THE INVENTION

The present invention overcomes shortfalls in the related art by presenting an unobvious and unique combination and configuration of methods and components to integrate eating utensils with hardware and software components to allow monitoring, analysis and sometimes control of human body functions.

The disclosed embodiments may be used with people at risk for or subject to:

Severe epilepsy and convulsions

Heart beat disorder

Alzheimer

Advanced aging

Children, specifically hyperactive children

Addicts

Acute mental problems

Bedsores

Disclosed embodiments may be used in several ways, including:

By online observation: installed on the user cell phones, the supervised person localization is done by APP. Also, the body, sitting, sleeping, walking, eating positions and online etc. are determined.

Related to the system: tin which the daily or weekly routines activities of the person are specified by APP and if there is a significant difference in the mentioned activities with normal conditions, the required follow up will be carried out.

It should be noted since eating and drinking and consumption of drug can be done concurrent with other activities these activities should be considered as a dominated activity at any moment, it is not considered a concurrent activity. With the table of FIG. 2, the state of day and weekly can be controlled.

The system has a database which for each eight applications mentioned above, there are many applications and normal model that are defined by a specialist of one or several activity model for the person and if the activity and if the activities, rest and stillness of the person and position and speed are not conformed to the selective normal models, the required warns are sent in three levels:

Warning to the person can be included a patient, an old aged or each person mentioned above.

Warning to The nurse (in the case of having a nurse)

Warning to the controlling person or responsible one including family, the head nurse, hospital ward and company control health and etc.

These and other objects and advantages will be made apparent when considering the following detailed specification when taken in conjunction with the drawings.

### BRIEF DESCRIPTION OF THE DRAWINGS

FIG. 1 depicts a table of rest and activities over a period of 24 hours

FIG. 2 depicts a table of rest and activities over a period of seven days

FIG. 3A, 3B and 3C depict locations of x, y and z axes

FIG. 4 depicts a vector angle relative to the x and y axis

FIG. 5 depicts the installation of sensors on a human body

FIG. 6 depicts a block diagram of data transfer and analysis

FIG. 7 depicts various sleeping positions

FIG. 8 depicts a table regarding data of the sleeping positions

FIG. 9 depicts a block diagram measuring the transfer of acceleration to the main board

FIG. 10 depicts the placement of additional heart rate sensors

FIG. 11 depicts a block diagram regarding the measurements obtained from the sensors

FIG. 12 depicts a table of recorded sensor data

FIG. 13 depicts further sensors placed on a human body

FIG. 14 depicts a table of data regarding patient position in different situations

FIG. 15 depicts a block diagram of sensor measurements and GPS values

FIG. 16 depicts geographical boundaries for travel outside of a patient's house

FIG. 17 depicts interior geographical boundaries

FIG. 18 depicts a block diagram of data transfer to an APP

FIG. 19 depicts a block diagram of data transfer regarding abnormal movements

FIG. 20 depicts a table of data regarding a person at different time periods

FIG. 21 depicts data transfer of a disclosed embodiment

FIG. 22 depicts a block diagram of disclosed components

FIG. 23 depicts various means of data transfer

FIG. 24 depicts a system of data transfer

FIG. 25 depicts a utensil system of the prior art

FIG. 26 depicts a disclosed interface and icon system

FIG. 27 depicts data recording and angles of use

FIG. 28 depicts a data connection with a spoon or other utensil

### REFERENCE NUMERALS IN THE DRAWINGS

1 a sleeping position as shown

2 a sleeping position as shown

3 a sleeping position as shown

4 a sleeping position as shown

5 a sleeping position as shown

6 a sleeping position as shown

7 a sleeping position as shown

8 a sleeping position as shown

9 a sleeping position as shown

10 a sleeping position as shown

11 a sleeping position as shown

12 a sleeping position as shown

### DETAILED DESCRIPTION OF EMBODIMENTS OF THE INVENTION

The following detailed description is directed to certain specific embodiments of the invention. However, the invention can be embodied in a multitude of different ways as defined and covered by the claims and their equivalents. In this description, reference is made to the drawings wherein like parts are designated with like numerals throughout.

Unless otherwise noted in this specification or in the claims, all of the terms used in the specification and the claims will have the meanings normally ascribed to these terms by workers in the art.

Unless the context clearly requires otherwise, throughout the description and the claims, the words “comprise,” “comprising” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “above,” “below,” and words of similar import, when used in this application, shall refer to this application as a whole and not to any particular portions of this application.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not only the systems described herein. The various embodiments described herein can be combined to provide further embodiments. These and other changes can be made to the invention in light of the detailed description.

Any and all the above references and U.S. patents and applications are incorporated herein by reference. Aspects of the invention can be modified, if necessary, to employ the systems, functions and concepts of the various patents and applications described above to provide yet further embodiments of the invention.

These and other changes can be made to the invention in light of the above detailed description. In general, the terms used in the following claims, should not be construed to limit the invention to the specific embodiments disclosed in the specification, unless the above detailed description explicitly defines

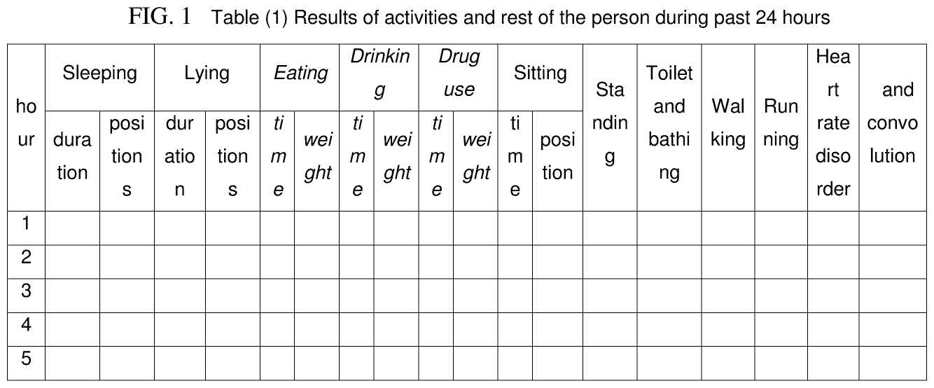

FIG. 1 depicts a disclosed embodiment comprising a plurality of eating utensils integrated with various components that may include batteries, Bluetooth, software, a monitor/vibrator system, a scale, a microphone, an optional speaker system and other components.

An eating utensil may use a microphone to receive user information, such as food consumed or desired goals. An eating utensil may weigh food via an integrated scale. An integrated motion monitor or accelerometer may measure the movement of an eating utensil. An integrated vibrator may cause an eating utensil to vibrate and thus warn a consumer to stop eating, or at least check their current caloric balance.

FIG. 1 depicts a table of results of activities and rest for a person for a 24 hour period. A prior art system, such as Calorie Balance System U.S. patent a20150325142) is used for obtaining time, food weight and drinks date and the weight is measured using smart spoon (number 1) , fork (number 1) and glass (number 1) that measure the weight and consumed liquids in (FIG. 1 or Table 1).

For obtaining data on drug consumption, the pills box will be placed in the smart glass (number 2) and for measuring syrups, the smart spoon (number 2) for medicines consumption is used.

If the eating and drinking is considered, minimum 2 smart spoons (one for food and the other for syrups) will be used. Using system caring for people under surveillance and application for the people with epilepsy and convolution.

This system is used for people with epilepsy and convolution and the location, time, speed, acceleration and movement of the sensors on the clothes or body are measured. The warnings are announced, if the rest time is more than the determined time in the application and time models comparing the common places for rest such as bedroom, sofas and other places. Also, by examining the speed, acceleration and body movement with maximum defined speed and acceleration in the system, the probability of nerve and convolution attack is registered and these events are announced by warnings.

System for people with Alzheimer

Utilization of this system for the people with Alzheimer is allowable by supervision of the commuting, drinking sleeping, bathing and routines and timing of these activities and also drug use. On the other hand, controlling the daily activities such as eating, and are done. If the consumed food or drinks and other activities are less or more than allowable range and models, for the required warnings are announced.

The application of this system for people with bedsore, caregiving are done based on the comparison of received information on localization, time, speed and movement of the sensors on the body by the monitoring of defined models that provide the possibility of control of that person conditions during resting or wrong movement or wrong period in one position that the APP sends the warns as messages.

Monitoring and control of the children daily activities specifically children with mental problems .

Monitoring and control of the children daily activities specifically children with specific problems, the addicts activities and the people with mental problems are possible.

For example, the activity and speed of a child with specific problems are monitored and analyzed based on normal conditions and in the case of difference, the amount of eating and drinking is announced by using specific spoon, fork and glass (patent publication 20150325142).

People with mental and psychological problems

Similarly, monitoring and control are done in accordance with the time-tables by monitoring the amount of activities of localization, time, speed and acceleration of people with mental and psychological problems.

The base algorithm of this system on directivity the direction the body in X,Y,Z angles and subsidiary algorithm is speed, acceleration and GPS data.

The algorithm is different in sitting, standing, sleeping and each behavior follows its own algorithms. In sleep mode, the body is located at approximately one level and there is a slight distance in depicting the direction of the axes X, Y, Z as the height between the arms and legs and the body under observation for angles and the positive and negative positions of the organs of the patient's body show the main axis and the legs do not create a particular angles. If a person stands, then the hands and feet are not angular about the axes X, Y, Z they are roughly the same axis during sitting and other activities.

Thus, the position of the person in terms of physical state can be determined and then one can monitor himself using this method by revealing the sample of the placement of the axes of the hands and feet.

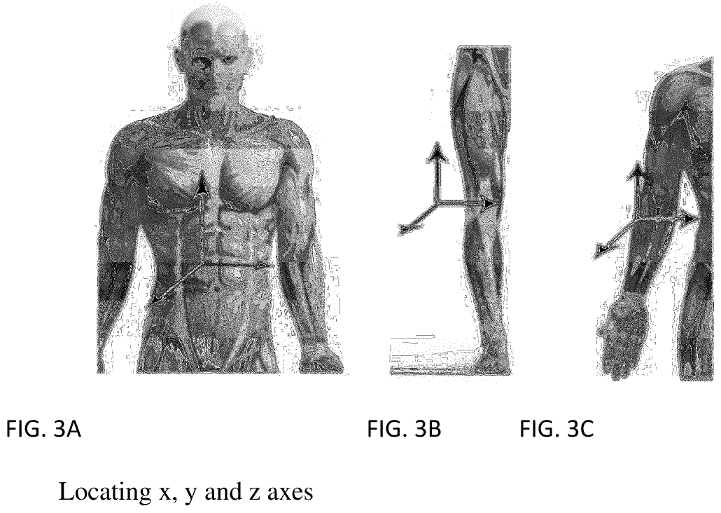

Referring to FIG. 3A, 3B and 3C, ﬀ the body of a person is considered and the hands and feet are imagined in a three-dimensional way, then for each hand and foot and the body axes, should be considered axes X, Y, Z Y, and left to Z-direction and to the X-direction. For each axis of the hand and foot in a three-dimensional angular deviation, the angles of each axis are taken into account and important information are shown about time of sleeping and directions of the body organs during sleep and in different moments, the position of each body in relation to each other and also during movements. FIGS. 3A, #B and 3C represent x, y, z. with this system can send position of body to table 1 online and show position and state of body in APP

A vector is considered for each direction in the figure and the positive and negative directions are considered and each position of the body organs is measured using these vectors.

There are different modules on the market that show the X, Y, Z vectors relative to each basic vector, then using and connecting them to the hands and feet and the body and prioritizing each of the movements and the axes at standing and sleeping and sitting, it can offer a correct picture of the general state of the person and the position at each moment is determined. Accordingly, there is an electronic circuit designed, and connect to all the sensors and analyze the sensor data online every moment.

In this invention, the various information anywhere and part in the body of the person under observation can be known.

The required information includes the position on terms of the angle of the body relative to the reference level and in the three x, y, z axes so that entire 360 degree angle on each of the axes conformed and measurable.

At each point and part of the patient's body, the angular velocity is calculated and it is determined at what point and at what speed this part of the body moves at any moment.

To measure this speed and direction of movement used. The modules have the ability to detect angular velocities. Angular velocities are calculated on the axes x, y, z and by combining the angular velocity, this velocity in spatial part of the patient body is obtained and from that movements are identified.

The speed angular speed at each point of the person under observation in each of the three axes is determined. In this case, the angles of acceleration or the first-order derivations are computed on each of the three axes x, y, z, and calculated. Using the derived velocity information at moments when the rate suddenly increases or decreases, it is possible to detect an unusual incident including neural attacks and epilepsy.

For each of the above functions, a separate module is created, but it is possible to select a module that describes all the movements or at least two of the specified movements and provides the necessary data in those parts. The reason for using this module is to match the data with each other and there is a common reference between them. For example, the derivative speed is the first order of displacement and the acceleration is the derivative of the second order of displacement. If the reference of the displacements is not specified, the acceleration and speed of each of the three axes are mistaken and lack the required accuracy. Therefore, in all parts of the system, for the functions to be identified, the modules are investigated individually of the monitoring system by the modules in each section.

The sensors are usually nine or more and in this system, there are nine sensors in the system mounted on the hands and feet, on the body as shown in FIG. 3A-C. The sensors at any time measure the degree of intrusiveness of the body relative to each axis (X, Y, Z) with numbers from 0 to 255 shown as 8-bit electronically. Then, the axis data is examined in a processor and the various states of standing and sitting are examined and analyzed. Block diagram may show the path of measurement, analysis and transmission of data to the original board.

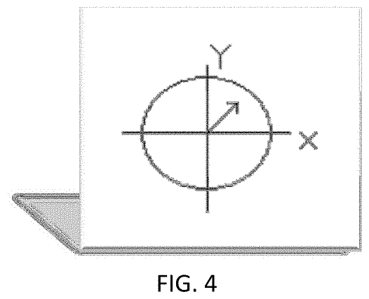

In FIG. 4, the angle of the vector relative to the axes x, y is approximately +45 degrees and in quantitative terms, for the reported examinations it is about 64.

For the construction of the circuit, the sensors representing the angles x, y, and z are used. These sensors exhibit values of 0 to 255 at their angles from 0 to 360 in their output and in a multi-chip module consists of two integrated molds in a single package. The body of the gyroscope is 3 axial.

To have connection with all the equipment registers using high speed 120 communication controls, it is used for speeds up to 20 MHZ SRI communication methods. The sensors should have a high resistance to heavy shots. The nine sensors that used in this device are used on two scapula and two legs and one on the chest.

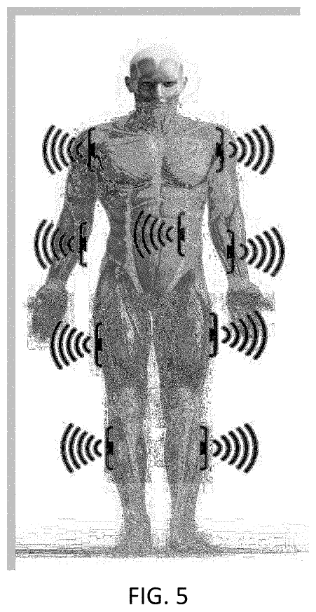

FIG. 5 shows the attachment of sensors on a body. These sensors monitor all body parts and show body the standing, sitting and sleeping set to body to face and back.

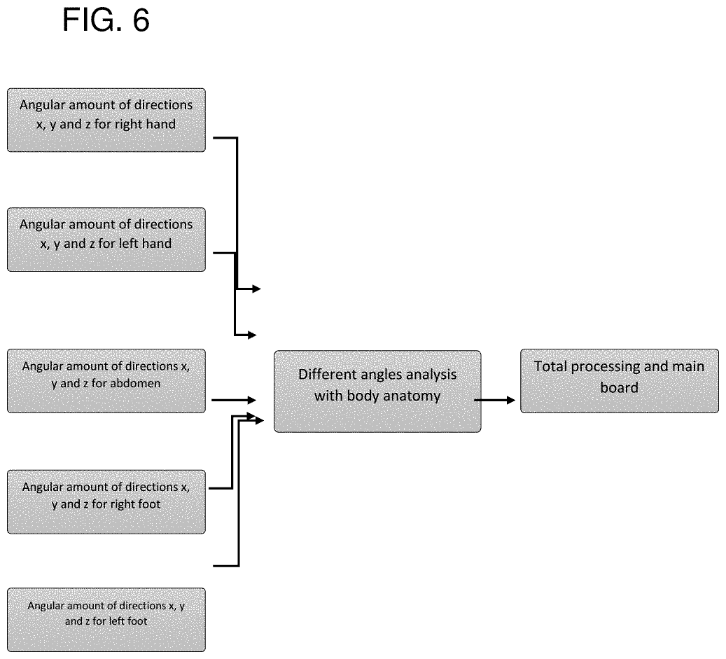

FIG. 6 depicts a block diagram for the measurement, analysis and transfer of collected data. Angular velocity and movement of each body parts can be detected using gyroscope sensors and with separate use in each part of the body, in addition to showing the position, it can be shown the direction and speed of movement.

Control and check the positions of the body during resting and the time and place activities and body movement of patients with epilepsy and/or convolution and re disposing bedsore lesions

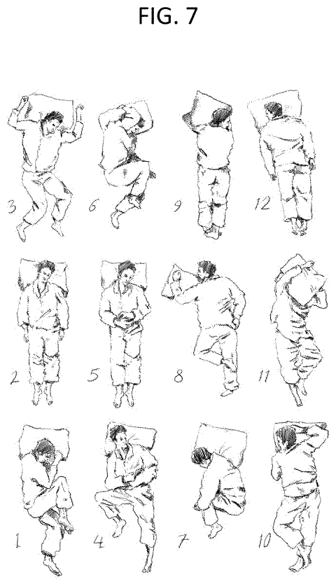

Referring to FIG. 7, in a disclosed embodiment, more than a dozen models of sleeping patterns in database have been defined. There are other models for sleeping and the system is able to extend the sleep states when it defines the others and has more complete results. In FIG. 7, several examples of sleep patterns have been shown as examples.

In state number 2, the angular size of the axes (x, y, z) for the left and right shoulders of the right and left legs and the right leg are {(−30<x<30) and ((−30<y<30) {−30<z<30); if in the sleep mode, the body is at position 12, the axes (x, y, z) for the left and the right shoulders are {(−30<x<30) and (−30<y<30 and {(−240<z<−300) and for the left foot {(−30<x<30) and (−30<y<30) and (−110<z<90)}. In sleeping mode 1, the vectors for the left and right and the left shoulders are |{(−110<x<90) and (−30<y<30) and (−240<z<300). In Table 3, for all 12 states, the angular value is shown.

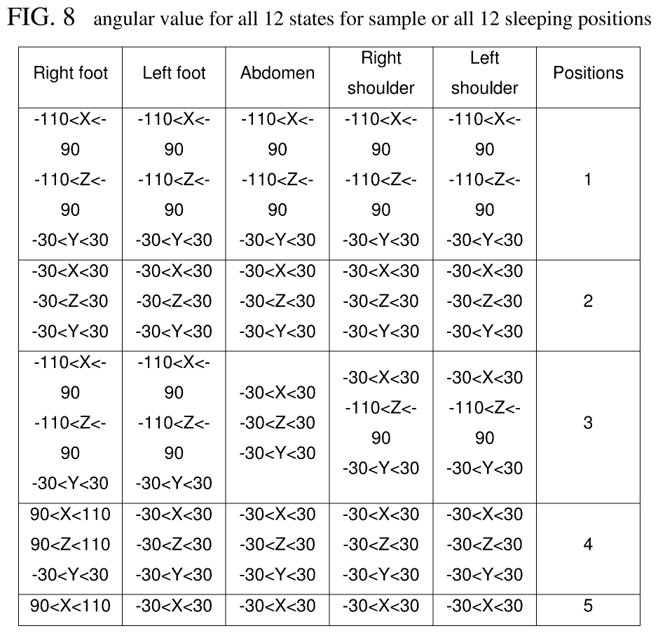

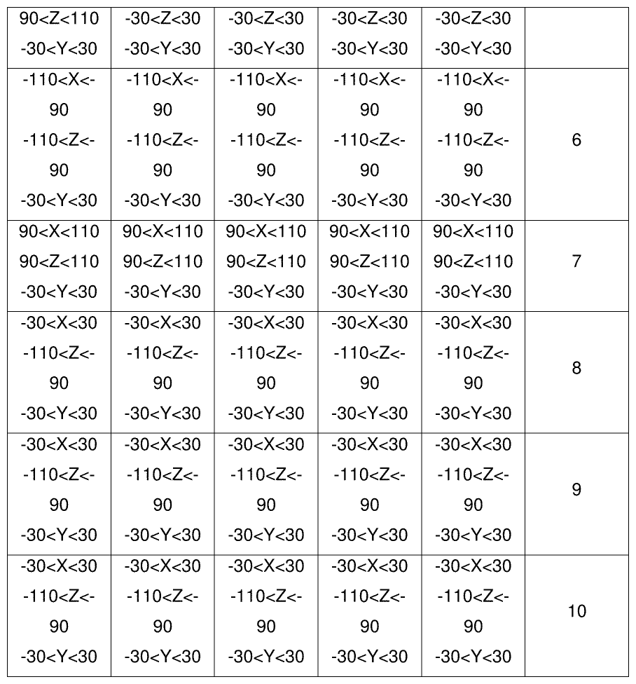

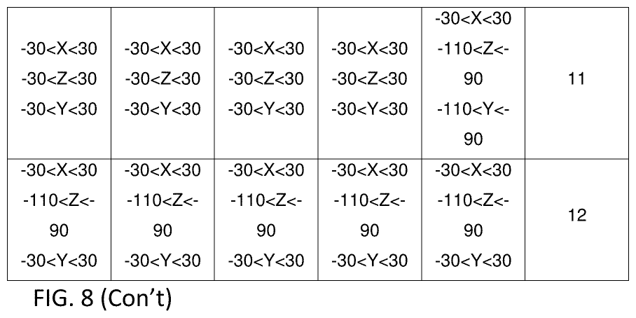

In FIG. 8, angle values are shown for all twelve sleeping positions.

For each position, the size of the necessary vector intervals are extracted using vectors and different types of states and the carpenter installs the system on the device by comparison of the appropriate models and positions with the appropriate selected application and models.

After revealing the size of the x, y, x axis of the hands, feet and the body and manner of locating and schedule changes in axis and position of the body, if the specified time interval and the axis are not changed, in other words, the person sleeping mode does not change, the nurse or any other care taker person will be announced with the use of sms or audio warnings and the nurse would take an action within the specified time period for the desired and defined movements otherwise, after the second time, the relatives will be notified via the alert message to the APP installed on the mobile.

All images are in the still body state and the output data from the gyroscope or the same angular velocity for each of the axes is equal to zero and the acceleration in each axis is equal to zero or at least an amount that only indicates the body's vibration in the normal state.

Acceleration equals the rate of change in speed divided by the time unit. The accelerometer is used to detect the speed of movement and how to change the movement of the x, y and z axis to each part of the body.

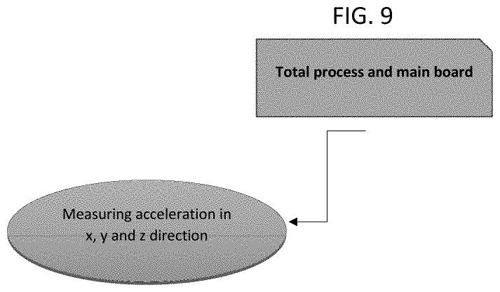

In this system, three-axis accelerometers are used which it is a small, 3-axis low-power accelerometer whose output voltages are exactly the same (able to measure up to 3 g, as the acceleration of the earth) that measures dynamic (vibration) and static accelerations (like ground acceleration). The output of the three voltages is variable which can be directly measured by the processor. FIG. 9 shows the measurement and transmission of acceleration data to the original board.

The adjustable processor widths range from 0.5 Hz to 1600 Hz for X and Y axes and 0.5 Hz to 550 Hz for the Z axis. The sensor is mounted on the chest or back. For proper use of this IC, a system is placed in the person's abdomen to show the overall speed of the person in different directions.

In FIG. 9 a block diagram shows a system of measurement and transfer of information to a main board.

Controlling and examining the trend of heart rate and hearth in people under the risk of stop or deficit of normal heart rate

One of the features of the device is the presence of a heart rate sensor and bedsore and being within the normal range that can be measured online.

If the heart rate is not in the normal range, the device alert will report a hazard and signal, the next step, if it continues in addition to the nurse or caregiver after the prescribed period, relatives will be informed.

In the case of rest and heart failure, this device alerts and warns in an emergency. In this case, there is a difference between stopping of the sensor by the person or a nurse on the heart failure. The heart rate sensor is used in this system to record heart rate online and in the case of registration, it will alert the controlling person the state more than a standard or lower standard.

This sensor can measure the pulse of the person easily and determine the pulse rate by touching the wrist or neck vessels. The pulse sensor in each area of the skin reveals subtle changes in blood vessel dilatation to measure heart rate. The data pulse sends the controller to do processing.

The pulse sensor should amplify heart rate and eliminate its noise to provide easy and reliable pulse reading. This sensor can be launched at very low voltages. The current consumption of this sensor is low. FIG. 10 shows how the sensors are positioned in addition to the heart rate sensor. In block diagram 9 or FIG. 9, measurements of the values of all angular angle sensors, angular velocity, and heart rate and their transmission to the central range of the device are shown. In FIG. 8, the values of sensor data are shown in the main board and APP software.

FIG. 10 shows the placement of additional sensors.

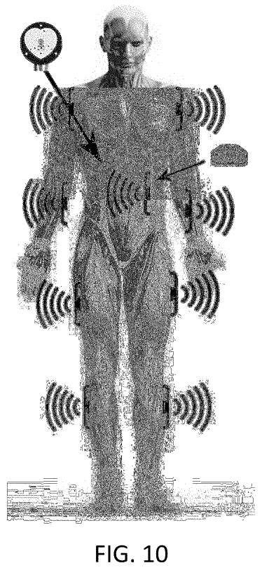

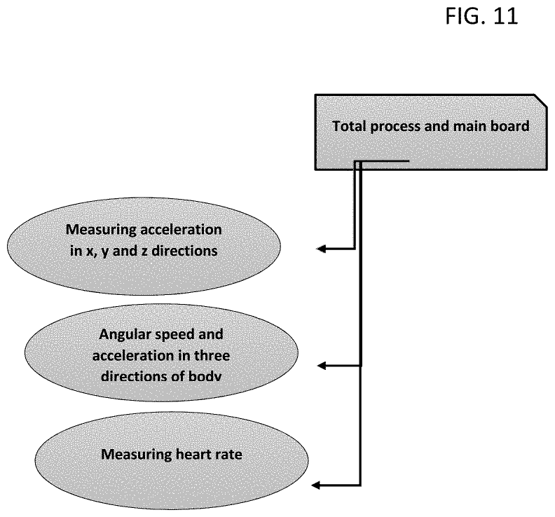

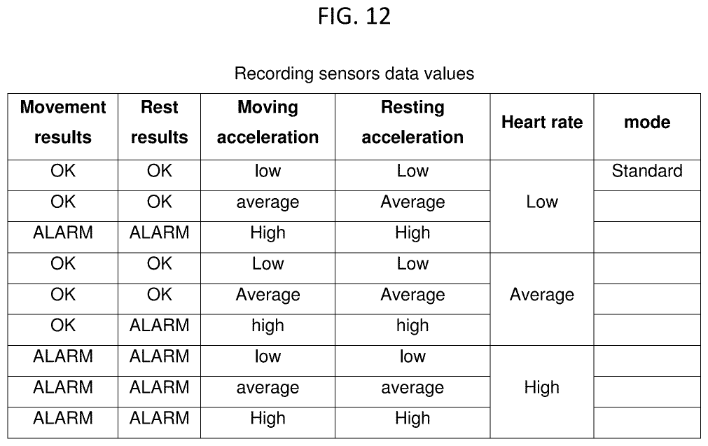

FIG. 11 or block diagram 11 shows measurement properties of the sensors. FIG. 12 or Table 12 shows the recording of data values obtained from the sensors.

Controlling and monitoring and behavioral analysis and control of the elderly and the young people and check their daily activities and Controlling and monitoring and behavioral analysis with Alzheimer

Controlling and analyzing the behavior of the elderly and underage people and knowing how they are doing.

To control the commuting of people, the device can be connected to the GPS so that it takes a range for the patient and if the patient is out of range, the device alerts the message.

The GPS module employs 12 satellites outside the atmosphere and extracts the location of the location using high-precision latitude and longitude vectors.

If the location of this device is changed, the new location will be extracted using the Google map software. By detecting the longitude and latitude, the location of the module can be identified. One of the uses of this module is to track a person or device that is connected using mobile lines or Internet.

GPS module data is sent to the supervisor by sending a sms packet of data and the controller will recognize the location of the individual or device by comparing data. This feature is one of the device options and alarms and information are provided by using the module and defining a functional range for the permission to travel to the patient, if the patient is out of range. In FIG. 6, the positioning of the sensors plus GPS positioning is shown.

The GPS module is used to show the person coordinates. In this system, a mobile-modular module is used that can connect to the mobile network and send an alert message using the SIM card in appropriate level. Meanwhile, it has the ability to communicate and transmit data and the ability to receive data from the satellite in difficult conditions. Table 5 shows the patient's position in different situations. In algorithm number (3) measurements of sensor values and geographic positioning system are shown.

If the person is inside the house, the positioning antennas should be used to examine the various aspects of the home and for the patient in different situations such as the bathroom, which is problematic for him, a warning message is required so that at the time of the GPS setup, the location map of the person under observation is declared to the device in order to define unauthorized or time-limited ranges. FIG. 7) shows the range specified in the location for the space out of the house and also FIG. 8) shows the range specified in the locator for indoor space.

In order to tuning the device inside the house, the first item is a complete map and if possible, a 3D map to the device memory as a layer system is necessary (layers are introduced in each layer of the map), for example, the general layout of the home and the second layer of the bed location and the third field are specified.

Using the access layers, the home position should be fully integrated with the monitored device in such a way that the person under observation (restricted) from the home environment will announce their alarms. Then the restricted areas are defined so that if the person goes to these areas inside the house, the device will create the necessary alarms, and in the next step, some of the equipment in terms of location should be defined. In other words, the location of the beds and the sofa and the chair should be separated so that the general shape of the body revealed by the software in different situations and times are detect the location of the patient will be accurately determined.

Other cases required by the nursing and controller opinion can be defined in the software so that the software can issue the necessary commands depending on the conditions and the manner in which it is placed and other things such as acceleration.

The device data is transmitted to APP software by various communication methods, where the final processing is performed. FIG. 11 shows how to transfer device data to the APP.

FIG. 13 shows the placement of additional sensors.

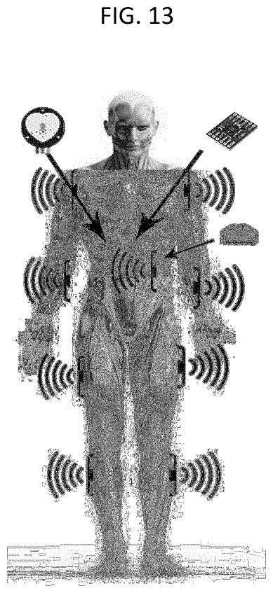

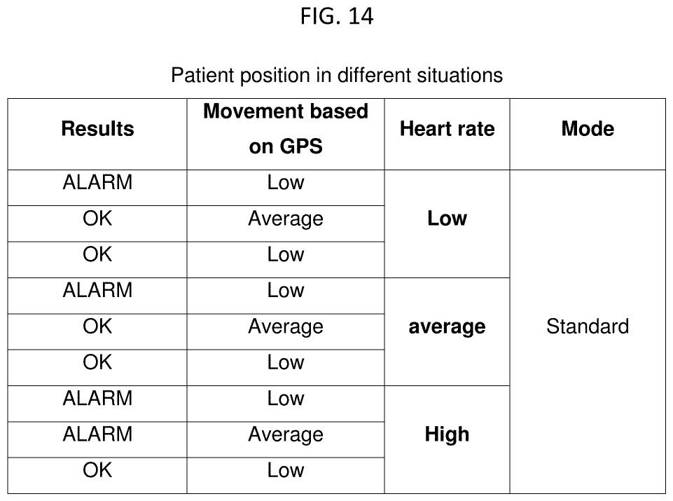

FIG. 14 shows various values for a patient in various situations.

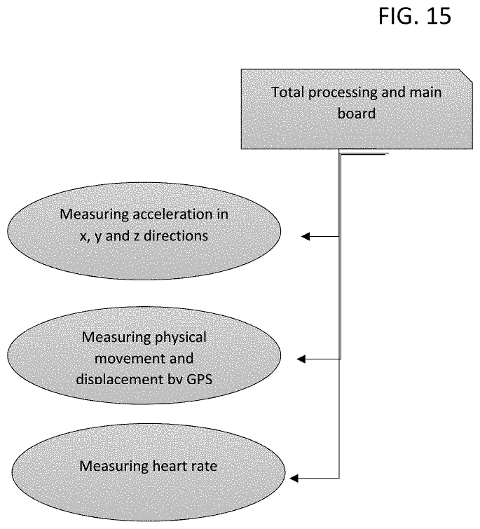

FIG. 15 presents a block diagram showing various measurements and data transfer.

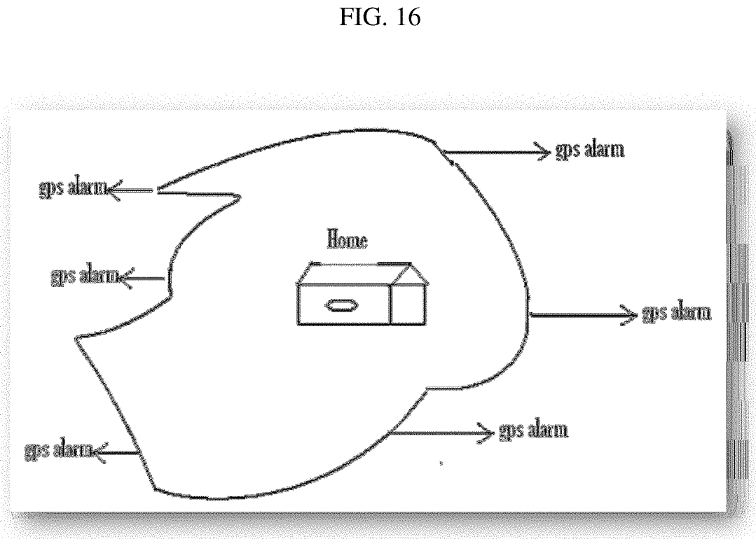

FIG. 16 shows GPS enabled boundaries around a home.

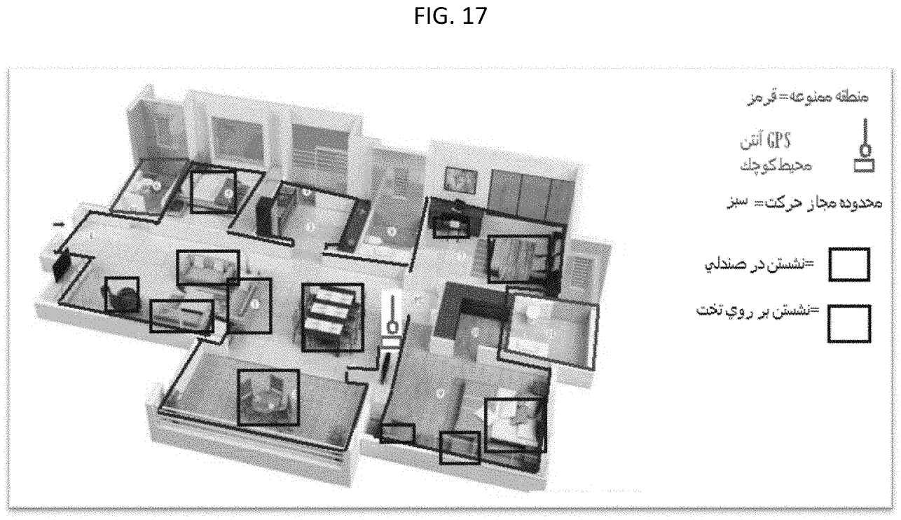

FIG. 17 shows forbidden areas within a home.

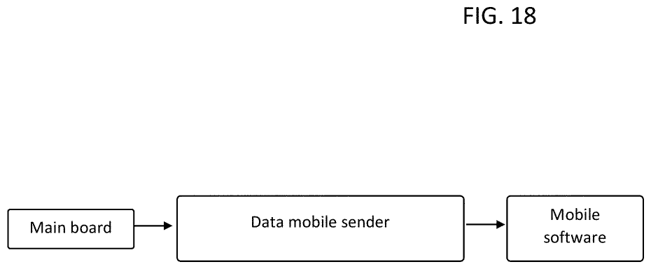

FIG. 18 or block diagram 18 shows the transfer of data from a main board, to a data mobile sender to mobile software

Control and examine the states and activities of addicted people or individuals with acute psychological problems

To control and monitor the states and activities of patients and addicted person by special planning of the device, at a specified time, rapid movements and vibration are performed. The device generates vibration by the accelerometer to the person controlling the risk of occurrence that indicates an abnormal occurrence of addiction in a person.

To control and evaluate the states and activities of patients with acute mental and psychological problems, changing from the defined mode to a greater extent than the time set in a person position performing a movement in a given time period that the behavior must be changed. Change in behavior should be within the permitted range of tolerance. In the future, the potential of adding artificial intelligence in order to analysis the results will be provided and more body states will be controlled by individuals. FIG. 15 shows how to transfer device data and how to process the attack trembles caused by abnormal movements of the addict' body

Control and inspection of the position of the rest of the body and of the time and place activities and the displacement of the body of patients susceptible to bedsore

In the case with a patient' susceptibility to bedsore, all individual behaviors, such as angles in different parts of the body and the angular velocity of each part of the body and the angular velocity of each part of the body and the way of rest and movement of the person should be monitored and warnings are given if the algorithm is specified and the time schedules for moving the body position are controlled by the selected models.

The mechanism of action is based on the fact that among the permitted rest and sleep modes a number is selected based on the physical characteristics and usual habits of sleeping and resting the person under observation. One person may be usually sleep for 5 positions and more often with another person. Based on this, not only the number of common modes and positions, but also several possible positions are selected from the table of different modes and positions of possible sleep. For each of the positions, the time interval is determined and the person under control should either change his body position or the nurse will change it . During this period, the nurse prevents the risk of developing the ulcer, and if this not happens, a warning system is required.

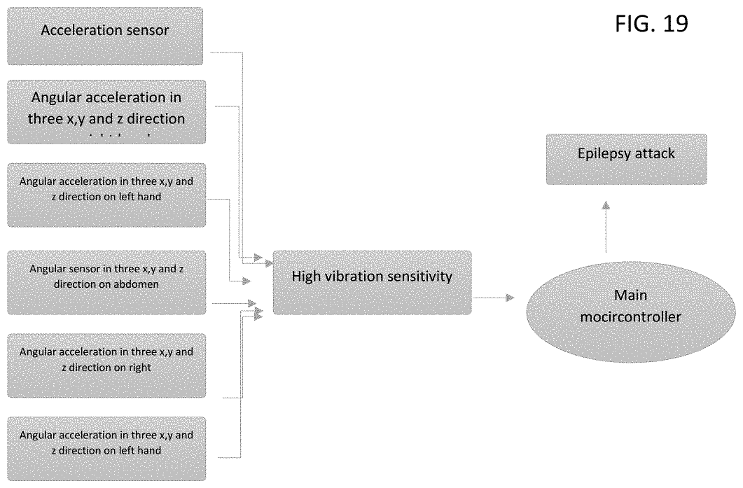

FIG. 19 presents a block diagram showing data transfer and processing for clarification of attack caused by abnormal movements of the addicted body.

Online display current status of patient and need help and model analyzed on this mode

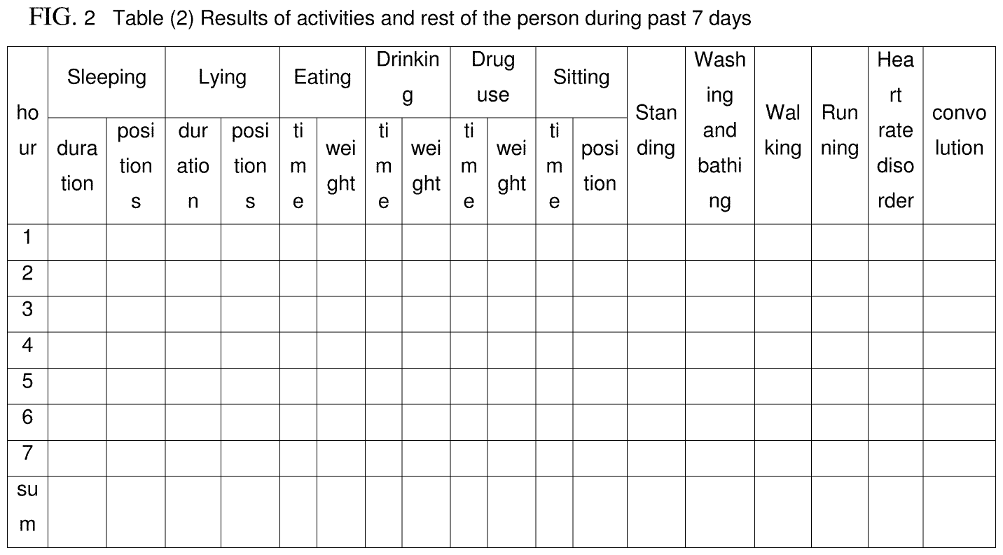

One of the most important features of the online screening device is the current state of the patient. All data received from someone's condition along with the diagnosis of the patient's current status is transmitted through a web site and a site defined in the software is stored in a database file and based on the information received at any moment as a sum of algebras of FIGS. 1 and 2 is completed and the user, using the site's address and password and patient name finds out about the patient's current status and the function of the device and the doctor can check the patient remotely by using the APP installed on the controller's handset at any moment visible and position of person to the two tables of FIGS. 1 and 2.

Saving the states and conditions of the patient and need help and ability to receive and analyze the person's previous condition

Due to the storage of current status data and the result of the behavioral analysis of the device and the physician's instructions and alarms, a person's history can be extracted and reviewed. A person's history is written in a database and stored in a site's web site and the scheduling and statuses and the time and the outcome of the behavioral analysis of the disease are deduced by the device as well as other algorithms for the diagnosis and subsequent decision of the physician or the controller.

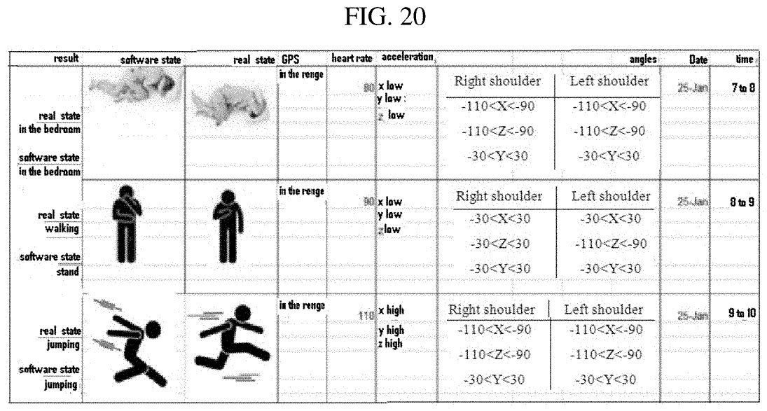

FIG. 20 depicts a person at different times of the day or night.

All person behaviors during 24 hours modeled using simulations and each of the individual states such as sleeping and sitting, reading, eating, drinking, walking and exercising and running, and so forth are shown.

The state table has no fixed time in any highlighting show of the time. In one hour, activities like going to the bathroom, sitting and showering can be done and recorded in two different times and recorded in the table. The recorded total time is 24 hours. For this purpose, some activities are prioritized, such as eating, bathing over other activities ,including sleeping, sitting .

If a person has behavioral changes due to epilepsy, using this method, epileptic seizures and a controller for better control of the disease and changes needed in the treatment process are presented.

According to the software analysis, the best form for describing the state that is under observation at that moment in that position is shown in such a way that the whole shape of the axes and their variations are close together.

In the manufacture of the device, hundreds of different modes and positions in different situations are distinguished from the view of the sensors used on the person's body.

In these hundreds of modes, the behaviors that are available for a move in different situations are reviewed with their changes in order to show the human desire for changes in the time interval as well as the person under observation. The events happened in bedtime are considered as a position state.

The overall function of the device is that in which 9 sensor blocks interact with each other, as well as with the mobile and related software. At any given time, online information is transmitted to the mobile device with full information and features and then the software written on the mobile phone uses the information given and the defined patterns to reveal the current state of the person.

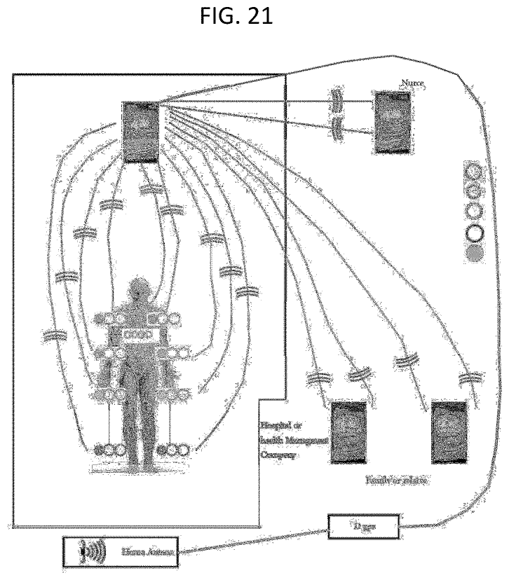

In the case of GPS and home antenna, the location information at any moment specifies the location of the person under observation is identified with the information obtained at each moment and the position of the person and the position of the person is controlled. Such processing has a very high level of processing amount and in all situations ,and the shape and position of the person is simulated. FIG. 21 shows general schema of the device and the way the data is transmitted between all parts of the device.

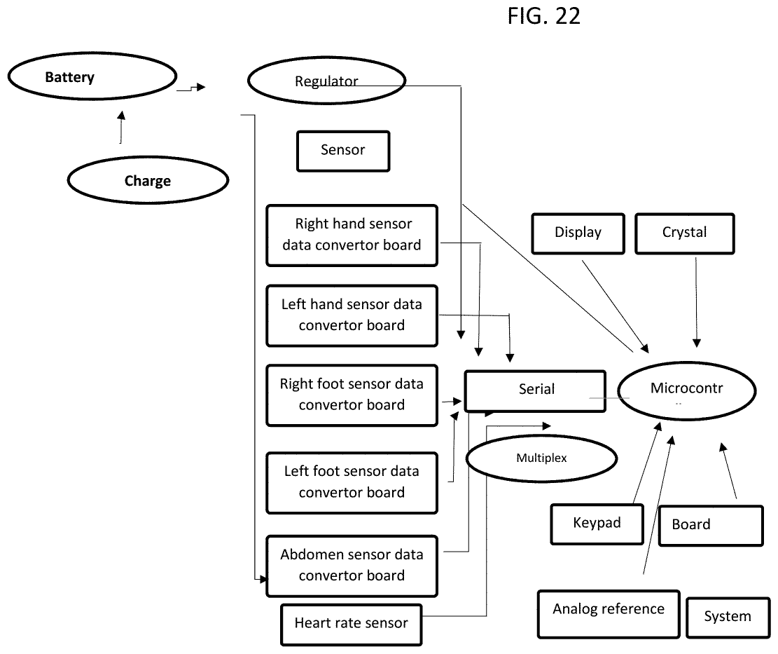

FIG. 22 shows various disclosed components and manners of connection.

The block diagram 6 shows the manner of connection.

The electronic circuit board circuitry of the sensors is in the form of (11). In this series, angular sensors are located in three directions x, y and z and a gyroscope sensor in three directions x, y, z and angular acceleration sensors in three directions x, y, z with a heart rate sensor.

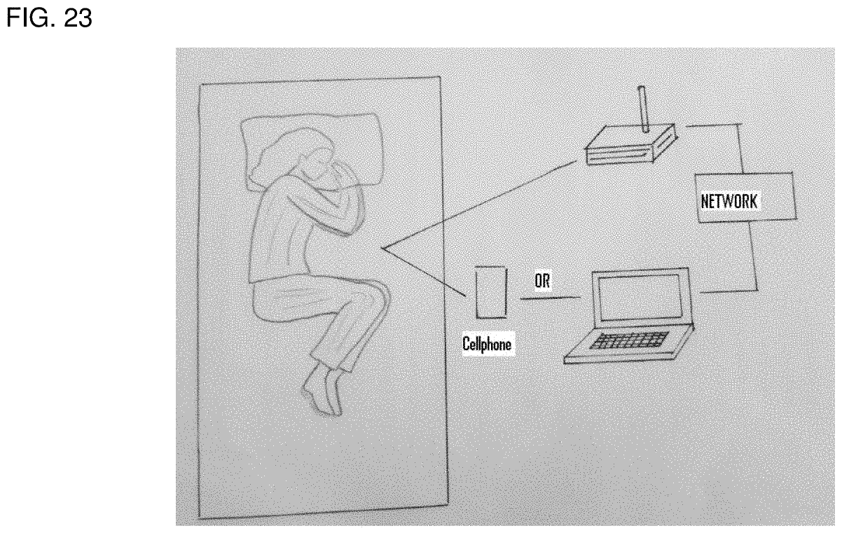

In this board, all 9 sensor wires connected to parts of the body are connected to each other and the data is transmitted to the mobile software after the initial processing by the WiFi system and the final processing and state detection is performed. By modem, the modem data is transmitted to any node in the world by the modem and is displayed in the destination computer of the processed data. FIG. 23 shows the device transmits data via a modem.

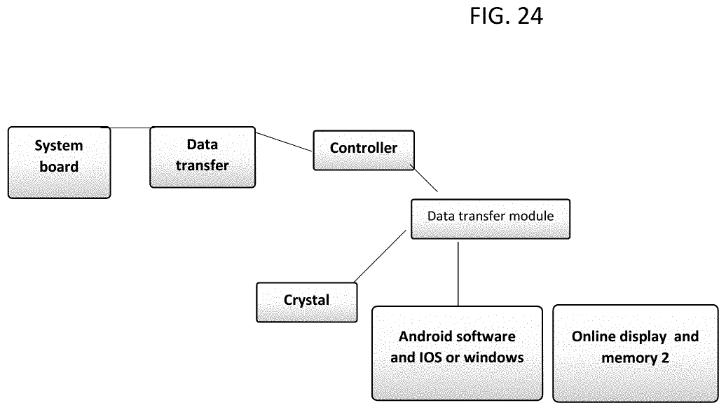

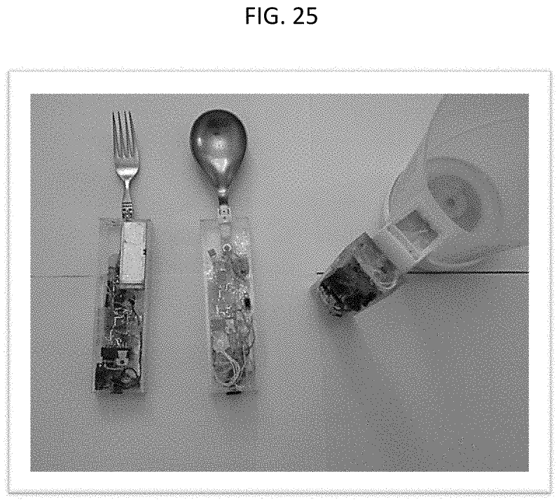

FIG. 24 presents a block diagram of data transfer to software from the original board.

The ability to display and determine the time and amount of food and time and drink consumed and the time spent on the drug by the person under observation and display it in FIG. 1

This system has the patent US 20150325142 and can be connected to the monitoring device. This device consists of a spoon, a fork and a glass. These devices have a measurement board and detect the data and it is connected wirelessly with wired or wireless connection to the main board or control software.

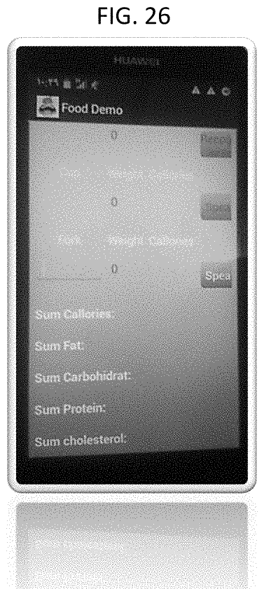

When the person wants to eat or drink or take medicine, they can begin by turning on the circuits connected to the spoon and forks and the glasses and the device shown in FIG. 26 automatically adjusts the amount of food consumed in terms of weight and time and submits the necessary information to the main device and specificities each of the spoons and glasses data related to meals, drinks and medicines consumed and other food specifications are registered in Tables of FIG. 1 and FIG. 2

For this purpose, the person under control should carefully insert the medications in a special glass of the drug and every moment when the box of drugs is withdrawn from the mug every day, the box will send out the box the daily drug in accordance with the change in the weight of the internal contents of the mug to the FIGS. 1 and 2 and for the medication that should be used by spoon no. 2 for the drug.

This system works so that there is a weight sensor inside each of the spoons, forks, or glasses and the weight of the food is calculated when the tabletop food is placed.

The weight sensor works on this device such as the Watson Bridge and displays the imbalance due to the presence of weight in one part with a small amount of voltage at the bridge output. Then, with an op amp amplifier with a high gain, the small voltage value of the weight sensor is measurable. The output of the op amp is transmitted to the adc input of the microcontroller and within the microcontroller the voltage is converted to a digital number and the digital weight of the food is extracted based on the particular table (due to the loss of the sensor's weight).

When the spoon is used by the person under observation and the spoon in his hand, 2 other data is also sent to the microcontroller automatically. 1. Accelerometer 2. The module of the angles x, y, z, which indicates the position of the spoon.

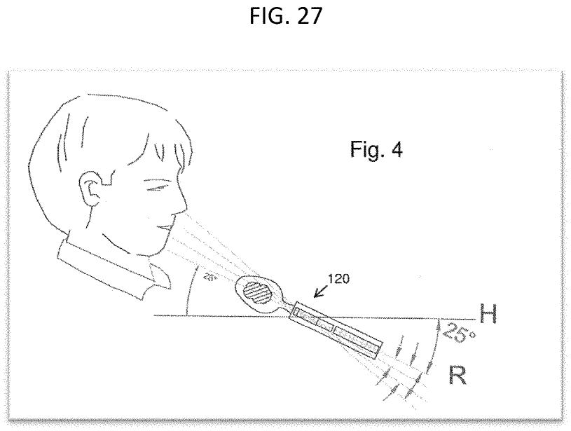

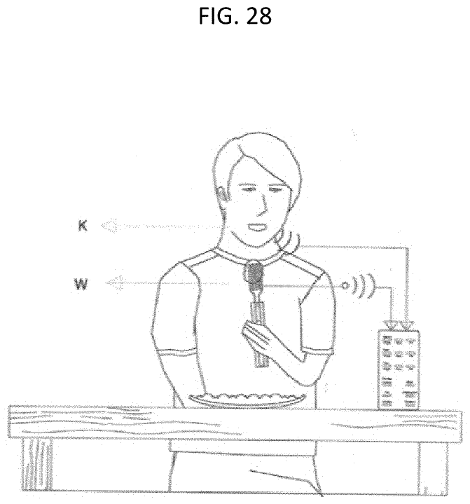

When the spoon was placed in front of the mouth as shown in FIG. 27 and FIG. 28 the connection between the spoon and the mobile application is established and the angular position between zero and 35 degrees relative to the surface of the ground, and the speed of the spoon movement becomes zero. In this case the weight of the food is measured and accumulated. The amount of food taken by the fork is also measured in the same way and is placed in the catalyst thereby determining the weight of the food and its time. In this way, the special spoon of the medicine sends the information to Table 1. The basis works for the drinking glasses and the glass for the daily delivery of the daily drug box. If the weight of the medication is low only the time of taking the medicine from the glass for the drug overnight is shown in Table 1.

FIG. 28 Displays the connection of the spoon with the mobile APP

The circuit made for the spoon and fork and the mug is connected to the mobile with the wifi module as well as the software for controlling the behavioral state of the body and the related software can also be added to the body's behavioral control software that shows transfer to the main software.

The alert is provided in the case of not eating or drinking fluids or drugs in accordance with diet plans or doctor's dossiers and food program, as well as non-observance of selected patterns and programs set by the person controlling the device.

Provide application software to show person's behavior at different times

All the behavioral patterns of people and individuals under observation, as well as the construction of the device for a particular purpose are carried out.

Putting the sensor on 9 parts of the body and recording the 6 part of each member relative to the axes x, y, z,

Detecting the angular velocity of each member of the body and determining which member moves at a moment.

Detecting the instantaneous acceleration of each member of the person's body to determine where the moment moves at which moment and whether it is in normal motion.

Examining the movements of individuals in hundreds of possible position positions in daily activities and identifying axes in each of their states

Behavioral analysis of each individual movement at certain times

Compliance with selected programs for health control for person's behavior during the day or night

Maneuvering a person's movement by detecting and identifying commuting places

Determining the person's heart rate to reveal acute conditions

Assessing the performance and implementation of the dietary instructions of the physician and the timing of the use of drugs

All of these items are in in-one health-care software provided by the device. It's available in Android or ios or Windows and runs on all mobile devices.

To enter this software, each controller who has given him the user name and password and the body (clothing) under the supervision of whom he or she carries the necessary sensors.

Medical orders are used as guides for the controller to perform at specified hours.

The software environment has 2 sections, one part for the monitoring mode as shown below

The males will be updated every second once and the patient information is stored in their data center and the nurse or physician or relatives of the patient can get information from the patient's condition by referring to the software at any moment and the person's behavior is also the programmed and updated.

The software is capable of controlling several patients at a time so that for each patient a block is registered and all the behaviors of the patient are placed in their block and displayed in the other block of the patient or the person in need of care. A software controller can monitor multiple individuals at a time, and if the device and system are used by the hospital nursing department or the relevant companies, there is no limit to the number of applications.

In other words, the device has the ability to connect to a control center, which can monitor to a very large number of people under observation and by checking the position of each moment, the person is informed of the state of each moment. Monitoring the status of boarding and checking for each person or persons under observation is done by reviewing Table 1 or 2.

Items.

The disclosed embodiments may include the following items:

1. A system for monitoring the human body, the system comprising:

a) a plurality of sensors, the sensors measuring movement and acceleration along the X, Y and Z axes of a patient, the sensors reporting measured data to a first mainboard;

b) a plurality of GPS sensors placed on the patient, the GPS sensors monitoring the movement and location of the patient; the GPS sensors reporting data to the first mainboard;

c) a plurality of GPS markers defining a perimeter the perimeter comprising a predefined containment area for the patient, the plurality of GPS sensors reporting data to the first mainboard;

d) the main board processing the reported data and outputting the patient's sleep positions, geographical movements, risk analysis for bedsores; and

e) the main board processing the reported data and outputting the patient's risk analysis for seizures and epilepsy.

What is claimed is:

1. A system for monitoring the human body, the system comprising:
a) a plurality of sensors, the sensors measuring movement and acceleration along the X, Y and Z axes of a patient, the sensors reporting measured data to a first mainboard;
b) a plurality of GPS sensors placed on the patient, the GPS sensors monitoring the movement and location of the patient; the GPS sensors reporting data to the first mainboard;
c) a plurality of GPS markers defining a perimeter the perimeter comprising a predefined containment area for the patient, the plurality of GPS sensors reporting data to the first mainboard;
d) the main board processing the reported data and outputting the patient's sleep positions, geographical movements, risk analysis for bedsores; and
e) the main board processing the reported data and outputting the patient's risk analysis for seizures and epilepsy.

2. A system used for treating people having one or more of the following conditions:
(a) high risk individuals before epilepsy and convulsion and severe
(b) at risk for heart failure or heart beat disorder
(c) individuals with Alzheimer
(d) old aged people or elderly
(e) children specifically hyperactive children
(f) addicts
(g) people with acute mental problems
(h) individuals suffering from bedsores
b) the system comprising: a plurality of sensors, the sensors measuring angle of X ,Y, Z and movement and acceleration along the X, Y and Z axes of a patient, the sensors reporting measured data to a first mainboard;
c) a plurality of GPS sensors placed on the patient, the GPS sensors monitoring the movement and location of the patient; the GPS sensors reporting data to the first mainboard;
d) a plurality of GPS markers defining a perimeter the perimeter comprising a predefined containment area for the patient, the plurality of GPS sensors reporting data to the first mainboard;
e) show and control and monitoring positions of the body during resting and the time and place activities and body movement of patients the states and tensions having psychological problems;
f) the main board processing the reported data and outputting the patient's sleep positions, geographical movements, risk analysis for bedsores;
g) the main board processing the reported data and outputting the patient's risk analysis for seizures and epilepsy;
h) online display of current patient status and saving the states and conditions of the patient.
